(19) United States
(12) Patent Application Publication
(10) Pub. No.: US 2021/0038827 A1
(43) Pub. Date: Feb. 11, 2021

(54) A CASING

(71) Applicant: DIABETES CARE TECHNOLOGY LIMITED, Ashington (GB)

(72) Inventor: Clive Peter Gunther, Haslemere (GB)

(21) Appl. No.: 16/976,016

(22) PCT Filed: Sep. 19, 2016
(86) PCT No.: PCT/EP2016/072209

§ 371 Date: Aug. 26, 2020

(51) Int. Cl. A61M 5/32; A61M 5/00

(52) U.S. Cl. CPC A61M 5/3202; A61M 5/002

(57) ABSTRACT

A casing is described for shielding a hypodermic needle. The casing has a protective rigid housing comprised of a first open end, rigid side walls and a second closed end distal from the first open end. The first open end of the housing includes a first cylindrical inner cavity, configured to receive a first end of a hypodermic needle supported by a needle mounting assembly. The second closed end of the housing includes a cylindrical end portion, the cylindrical end portion having a second cylindrical inner cavity with a diameter smaller than the diameter of the first cylindrical inner cavity, the second cylindrical inner cavity is configured to receive a second injection sharp end of a hypodermic needle, distal to a needle mounting assembly when the needle mounting assembly is received in the first cylindrical cavity. An outer wall of the cylindrical end portion further comprises a fixing mechanism for engagement with a clamping mechanism within a container. The fixing mechanism comprises at least one slot in an outer wall of the cylindrical end portion, extending in a direction perpendicular to the longitudinal axis of the first and second cylindrical end portions, the slot being configured for engagement with a clamping mechanism.

### CROSS-REFERENCE TO RELATED APPLICATION(S)

This application is a Section 371 National Stage Application of International Application No. PCT/EP2016/072209, filed on Sep. 19, 2016, entitled “A CASING” by Clive Peter Gunther, the whole disclosure of which is incorporated herein by reference.

### TECHNICAL FIELD

This disclosure relates to a casing for shielding a hypodermic needle. Certain embodiments of the disclosure are useful for assisting in the management of diabetes. They provide an improved design for a needle casing and a container in which to store the casing before and after the needle is used. Certain embodiments reduce the risks of accidents, as the casing cannot be accidentally removed from the container, and cannot rotate within it, making it difficult for the needle within to be screwed onto a pen. Additionally the casing can store used needles in a safe and secure way that allows for efficient disposal of the needles with low risk of injury.

### BACKGROUND

There are many situations and patients in the healthcare industry that require daily use of needle and other sharps. Professionals are trained in the safe storage, use and disposal of these sharps in order to reduce needle stick injuries. However there are many lay people, not medically trained, who handle needles on a daily basis. Those with diabetes fall into this group, as do patients suffering from any disease that requires regular, self-administered injections. These people are vulnerable to needle stick injuries, and it is desirable to try and reduce the risk of such injuries.

Diabetes is a common, life-long metabolic disease. It is estimated that there are 3.2 million patients in the UK alone. A person with diabetes cannot regulate the levels of glucose in their blood. This is controlled by a hormone called insulin, produced in the pancreas that allows the glucose to be used as fuel by the body. There are two main types of diabetes. In the first, Type 1 diabetes, the pancreas does not produce any insulin. In the second, Type 2 diabetes, not enough insulin is produced to regulate the levels of glucose in the blood, or the insulin produced is not effective as the cells of the body do not respond to it properly.

There is no medication to cure diabetes. It is a condition that must be managed by the patient every day. The most common medication used by a diabetes patient is insulin, to replace the naturally made insulin that the pancreas does not produce. Normal digestion prevents insulin being effective after ingestion, so it must be injected.

The average person with diabetes must inject themselves with insulin multiple times a day. Each injection requires a new, sterile needle. Each patient must therefore carry around with them many needles, and must have a safe way of storing them, attaching them to an insulin pen for use, and disposing of the needles after use. Especially as the patient gets older, dexterity reduces, so there is a demand for a simple, safe and sterile mechanism for storing, attaching and disposing of needles that does not require handling of the needles themselves.

Insulin injections are often seen as the most difficult aspect of a diabetes treatment plan to follow. The injection process is unpleasant, especially for children. Injection pens and pen needles are expensive, and the transport and storage of the pen needles is rarely convenient. However it is important to not only follow a treatment plan for the reasons set out above, but to administer the medication in a manner that is safe and hygienic. The risk of needle sticks when removing used needles from a pen is high. This is a particular risk for a carer, assisting in the delivery of the insulin, who should at no point come into contact with the end of a needle used on another person. Additionally, there is a risk that those with diabetes may attempt to reuse needles to minimize costs or limit the number of pen needles they are required to carry with them each day. This can increase the risk of those with diabetes developing lipohypertrophy, and should be avoided.

Healthcare professionals will be familiar with a variety of casings or shields for needles. For example the Hypodermic Needle-Pro EDGE®, manufactured by Smiths Medical provides a mechanism to cover a needle before and after use. However casings such as this one are not much larger than the long, thin needles they are designed to cover. This can make them difficult to handle, particularly for shorter needles. Although medical professionals may be practiced at using casings such as this one, those with diabetes, with no professional training, and often lower dexterity as a side effect of their disease, can find handling them difficult. It is desirable to provide a casing that is easily held, or securable in a larger more manageable container.

### SUMMARY

The present disclosure provides a casing for shielding a hypodermic needle comprising; a protective rigid housing comprised of a first open end, rigid side walls and a second closed end distal from the first open end, wherein the first open end of the housing includes a first cylindrical inner cavity, configured to receive a first end of a hypodermic needle supported by a needle mounting assembly, and wherein the second closed end of the housing includes a cylindrical end portion, the cylindrical end portion having a second cylindrical inner cavity with a diameter smaller than the diameter of the first cylindrical inner cavity, the second cylindrical inner cavity is configured to receive a second injection sharp end of a hypodermic needle, distal to a needle mounting assembly when the needle mounting assembly is received in the first cylindrical cavity, an outer wall of the cylindrical end portion further including a fixing mechanism for engagement with a clamping mechanism within a container, wherein the fixing mechanism comprises at least one slot in an outer wall of the cylindrical end portion, extending in a direction perpendicular to the longitudinal axis of the first and second cylindrical end portions, the slot being configured for engagement with a clamping mechanism.

A casing such as this provides a housing that is easier to handle for health professionals or lay people having to operate needles and other sharps regularly. The change in diameter of the housing provides a shelf that is easier to grip than a smooth, narrow needle casing. The single access point through which to remove or insert the needle minimizes the risk of needle stick injuries, as the needle is protected if even half covered. Furthermore, the casing provides means to allow the casing to be held in a container, larger than the casing, and therefore easier to handle. This may be particularly advantageous for lay people who must operate needles regularly, such as those with diabetes.

A large proportion of those with diabetes require regular insulin injections. This is usually performed using a reusable injection pen and removable, disposable needles. These needles must be stored in a sterile environment before use, they are usually provided in a sterile, sealed needle casing, and it is common nowadays and may be advantageous to have a mechanism provided with the needle for its safe removal from the pen after use to aid with disposal and minimize needle stick injuries. A mechanism that allows for simple storage and transport of multiple needles, and provides a mechanism to store used needles in a way they cannot be accessed for a second time reduces the practice of re-using needles and limits the risks of complications such a lipohypertrophy.

The rigid casing can advantageously provide the protection that a needle assembly requires. It must be sufficiently sturdy to protect the needle assembly from damage and allow an insulin pen to be screwed onto the needle assembly for attachment and unscrewed again after use. The casing must be able to support the forces applied during this connection. A single open end prevents the points at which dust and germs, etc., can enter the casing, hence the second end is permanently sealed.

Needles for injector pens are provided as part of a needle assembly, with a connecting end, including the thread for attaching it to the pen, and the needle extending from the other end. A casing must therefore have a first end wide enough to receive this connecting end at least. In the present disclosure, to ensure the needle is secure, the casing enclosing the needle end of the needle assembly is narrower than the first end.

Needle casings are usually sold alone or in combination with other identical casings. They can be used alone, with a user holding the casing between their fingers when screwing the injection pen into the connecting end of the needle assembly. However the risks of needle sticks, or accidents such as dropping the casing are high if using the casing in this manner as they are small and difficult to handle. It is common that a user has impaired dexterity or diabetic neuropathy, and therefore requires a device that is ergonomic. It may therefore be advantageous to provide a means of allowing the casing to be retained in a larger container that will hold it securely such that a user can hold the larger container with more ease and reduce accidents or needle sticks. A fixing mechanism may therefore be advantageously added to the outer wall of the casing to aid the casing being retained in a container. The most common form of retention is clamping. A clamping mechanism is easy to operate and simple to manufacture. Therefore a fixing mechanism to aid clamping may be an advantageous addition to the casing. In particular, a slot or groove that allows a clamp to fix more securely onto a casing will prevent a smooth edged casing being accidently pulled out of a clamp when the injector pen removed the needle assembly from inside. A casing retained in this way can provide a ‘touch-free’ mechanism, as at no point does the user needle to come in direct contact with the needle in order to use it.

In certain embodiments, the fixing mechanism comprises two slots in an outer wall of the cylindrical end portion, both extending in a direction perpendicular to the longitudinal axis of the first and second cylindrical end portions.

A clamping mechanism generally has two elements, and a slot to engage with each one provides more secure gripping onto the casing. The longitudinal positioning is the most secure arrangement to prevent the casing being accidently pulled in the same direction as the needle from the casing.

In certain embodiments, the fixing element includes two parallel slots on opposite sides of an outer wall of the cylindrical end portion, both extending in a direction perpendicular to the longitudinal axis of the first and second cylindrical end portions.

As a clamping mechanism is generally formed of two elements opposite each other, coming to meet in the middle, it may be advantageous to position the slots at points where they will meet the arms of a clamp.

In certain embodiments, the outer wall of the rigid housing includes a frustroconical portion connecting the first open end of the housing and the second closed end.

This smooth transition between the wider first end that receives the connecting end of the needle assembly and the narrow second end that receives the needle aids manufacturability and allows for simple injection molding of the casing.

In certain embodiments, the second cylindrical cavity comprises a narrowing to create a needle or sharps cavity with a diameter smaller than that of the second cylindrical cavity.

In certain embodiments, a needle sheath, covering a second injection sharp end of a hypodermic needle, distal to a needle mounting assembly supporting the needle has a outer diameter larger than the inner diameter of the needle or sharps cavity such that the needle sheath cannot pass into the needle or sharps cavity.

This is to create a shoulder at the narrowing that the needle sheath abuts when the needle assembly is inserted into the casing before use. When the sheath abuts this shoulder it cannot move in any further and the connecting end of the needle assembly is held at a position in the casing from which it can be removed by an injector pen. However after the sheath is removed during use, the bare needle is sufficiently narrow to pass into the needle cavity and the connecting end of the needle assembly can pass further into the casing.

In certain embodiments, the first open end of the protective rigid housing includes a projecting edge with a beveled first surface and a flat inner surface, extending radially inwards from at least one point on the circumference of the first open end.

In the embodiment shown in the figures, at least two of these edges are shown projecting at opposite sides of the open end of the housing. In other embodiments, the present disclosure would work if there was a single projecting edge, projecting from a single point on the open end of the housing, or if the edge extended the full way around the circumference of the open end.

In certain embodiments, the needle mounting assembly has a diameter larger than the narrowest diameter of the projecting edge, such that it cannot pass over the flat inner surface of the projecting edge.

This arrangement means that after use, when the needle of the needle assembly is bare and can pass into the needle cavity of the casing, the connecting end of the assembly can pass far enough into the casing to pass over the projecting edges and be caught under them. This prevents removal of the used needle. The used needle is then permanently held in a casing with a closed second end which eliminates the risk of accidently needle sticks. The casing can then me disposed of with the used needle inside.

In certain embodiments, the casing includes a removable lid to seal the first open end of the housing.

The lid is first applied during manufacture to seal the needle in a sterile environment. It is easily removed by the use to access the needle assembly for attachment to an injector pen.

In certain embodiments, the second closed end of the housing includes a recession or socket.

There is a problem with casings in a container that when the injector pen is screwed into the connecting end of the needle assembly, the entire casing can rotate within the container to prevent effective attachment of the needle to the pen. The slot engagement with a clamping mechanism may prevent this, but more effective would be a socket and plug arrangement at the base of the casing designed especially to prevent rotation.

Therefore sockets may be provided at the base of each casing in the present disclosure.

In certain embodiments, the socket is non-circular. In order to prevent rotation, the socket and plug arrangement cannot be circular, as then the plug would just rotate within the socket.

In certain embodiments, the socket is substantially square. A square is a reliable shape that does not allow rotation and is simple to manufacture, however it is appreciated that any shape that was not substantially circular would also work. A shape with at least one corner would be most effective.

In certain embodiments, the present disclosure further provides a container for storing and/or shielding a needle casing for a hypodermic needle, said container including a rigid first wall and a rigid second wall opposing the first wall, the container further including a compartment for storing said needle casing, defined by the first and second walls configured to be end walls, a front wall and a rear wall extending between the first and second walls, an inner needle casing support structure, a pivotable access door and a pivotable clamping element; the clamping element including a first end portion and a second end portion, wherein the first end portion is pivotably connected to a wall of the needle container element and the second end of the clamping element comprises a substantially U-shaped clamp, such that the clamping element is pivotable from a first position in which a needle casing within the needle container element is not in contact with the U-shaped clamp, to a second position in which the U-shaped clamp is engaged with the needle casing.

As discussed above it may be advantageous to provide a container into which a needle casing can be clamped. It is larger than a needle casing on its own and therefore easier to hold, which may reduce accidents during use. The container may be rigid to prevent damage occurring to the casings within it. There must be a way for a user or an injector pen to access the casing within it, but it to also be sealed, hence a pivotably door is used. Also, as discussed above, a clamping mechanism provides a secure attachment means for the casing within the container. The larger the surface area connecting the clamps to the casings, the better grip the clamps will have on the casing. As the casing is being clamped around a cylindrical lower portion, U-shaped champs that have a concave inner surface to engage with the curved outer surface of the casings may be advantageous. A pivotable clamping element is preferably to a completely removable clamping element as it eliminates the risk of losing the clamping element.

It is desirable to provide a way for a user or an injector pen to access the casing within it, but it to also be sealed, hence a pivotable access door. This is pivotable in the embodiment described in detail below for the same reasons that the clamping element is pivotable, but it need not be. For a needle container the access door is opened to insert the needle casing into the compartment, but also to access the needle itself. An injector pen can be inserted through the access door into the top opening of a commercially available needle casing. It can screw onto the needle, the needle can be used, and the needle can be returned to the casing for storage before disposal. The access door can enclose the needle casing even after the needle is used, providing a convenient and compact storage device that can retain the used needles until it is convenient to dispose of them.

In certain embodiments, the inner support structure includes a receiving surface for receiving a needle casing.

This is because, if there is one clamp on one clamping element, the casing should abut a surface on its opposite side such that it is secured in place. If the casing can be clamped against a receiving surface then there is only need for a single clamping element for the opposite side.

In certain embodiments, the receiving surface is substantially U-shaped.

As discussed for the clamping element above this increases the surface area in contact with the casing and therefore the strength of the clamp.

In certain embodiments, the clamping element includes an outer face that forms part of an outer wall of the container when the clamping element is in a first position, and an inner face that form part of the inner wall of the container when the clamping element is in a first position.

In this arrangement the clamping element forms part of the walls of the container. It may be advantageous to have no permanent openings in the walls of the container, but to also not have an unnecessary number of elements to manufacture. It may be advantageous that the clamping element serves both purposes.

In certain embodiments, the clamping element includes a projecting arm, extending substantially perpendicularly from the inner face of the clamping element.

This projecting arm can extend into the container to reach the casing within. This allows the main body of the clamping element to be positioned as part of the outer wall of the casing when in the first position while still holding the casing in place.

In certain embodiments, the at least one U-shaped clamp is positioned on the projecting arm.

In certain embodiments, the container includes a first substantially U-shaped clamp on the clamping element and a second substantially U-shaped clamp on the inner support structure.

In this arrangement, only one part of the overall clamping mechanism needs to be movable, simplifying the design of the container, but the container is still held in secure attachment within the casing from both sides over a maximum surface area.

In certain embodiments, the second substantially U-shaped clamp is positioned for engagement with a needle casing at point opposite the point at which a first U-shaped clamp on a clamping element engages the needle casing.

This positioning allows the clamping forces to act inwards onto the casing to grip the casing in the container from the same height at each side. This is the strongest arrangement for the clamps. If the clamps were at different heights the forced may act to damage the casing.

In certain embodiments, the at least one U-shaped clamp is formed of rubber.

Rubber would grip the casing most strongly due to the high coefficient of friction it exhibits.

### BRIEF DESCRIPTION OF THE DRAWINGS

An embodiment of the present disclosure will now be described, by way of non-limiting example only, with reference to the accompanying drawings, in which:

FIG. 1 illustrates an exploded view of a basic needle casing and the needle assembly to be held within it;

FIG. 2a illustrates a cut-through view of the casing of FIG. 1, holding a needle assembly before use, and FIG. 2b illustrates a cut-through view of the casing of FIG. 1, holding a needle assembly after use;

FIG. 3 illustrates a perspective view of a needle casing according to FIG. 1;

FIG. 4a illustrates a perspective view of a container for a needle casing according to FIG. 1 from the front, and FIG. 4b illustrates a perspective view of the container of FIG. 4a from the rear;

FIG. 5a illustrates a rear view of the container of FIG. 4a, and FIG. 5b illustrates a front view of the container of FIG. 4a;

FIG. 6a illustrates a first end view of the container of FIG. 4, and FIG. 6b illustrates a second end view of the container of FIG. 4a;

FIG. 7a illustrates a perspective view of the container of FIG. 4a connected to other containers and an empty container with pivotable access doors, and FIG. 7b illustrates a rear view of the containers of FIG. 7a;

FIG. 8 illustrates a cut-through of the container of FIG. 4a with an open clamping element;

FIG. 9 illustrates a perspective view of the cut-through of the container of FIG. 8;

FIG. 10a illustrates a needle casing within a container from the front with an alternative clamping mechanism, and FIG. 10b illustrates a cut-through view of the container of FIG. 10a;

FIG. 11 illustrates a cut-through view of the container of FIG. 10a with an open clamping element; and

FIG. 12 illustrates the container of FIG. 10a with an open clamping element.

### DETAILED DESCRIPTION

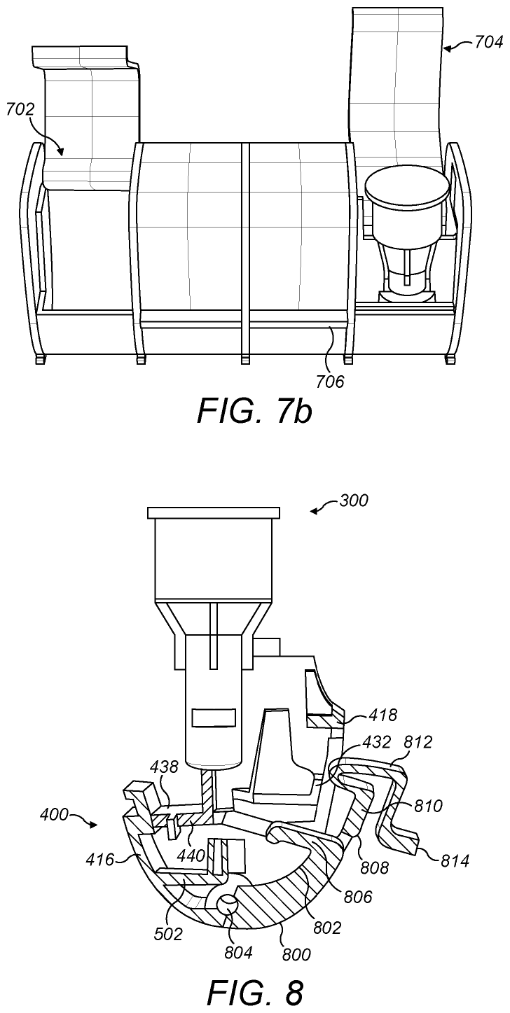

The words top, bottom, up, down, side, end and similar positional language has been used throughout this description according to the relative positions of features when embodiments of the present disclosure are orientated in the position of the embodiments shown in FIG. 7a, wherein the rear of the container is shown facing left. It is to be understood that the device may be used, stored and transported in any orientation, not just that shown in the figures.

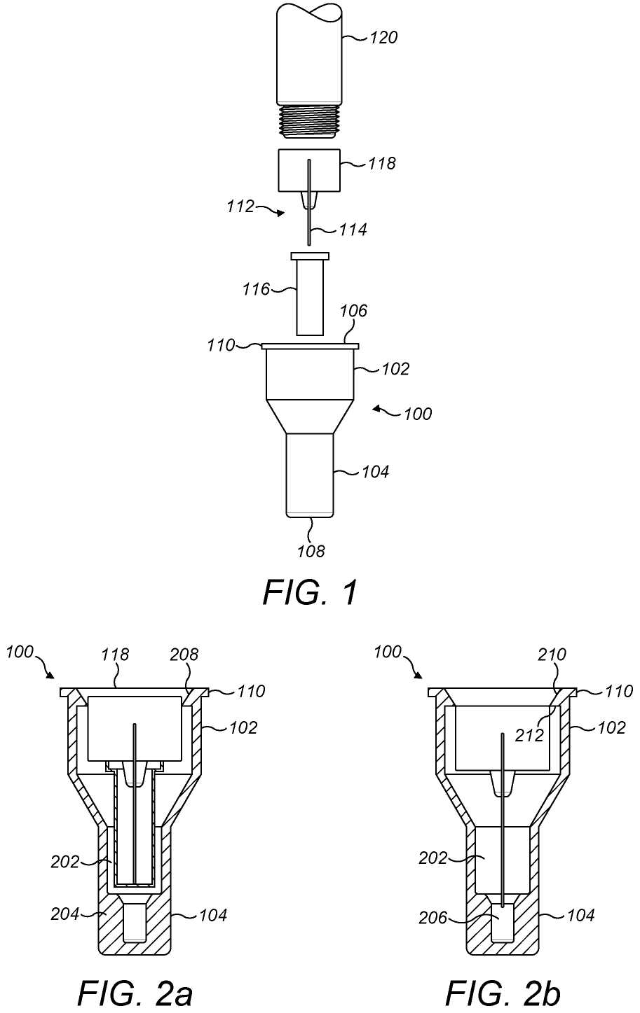

FIG. 1 shows a needle casing 100. It is generally cylindrical in shape and made of rigid plastics such as Acrylonitrile butadiene styrene (ABS). Each casing has a wide portion 102 separated from a narrow portion 104 by a narrowing in the middle of the casing. The wide portion 102 is open at a first end 106 of the casing, and the narrow portion 104 is closed at a second end of the casing 108. The wide portion 102 has a lip 110 extending around the circumference of the cylindrical casing at the open first end 106. This lip 110 provides a flat upper surface onto which a removable lid (not shown) can be attached. This lid is a flexible lid made of a material such as plastic or paper or foil that seals the needle into the sterile environment of the casing and is easily removed by a user before use.

The needle assembly 112 to be received by the needle casing 100 comprises a hypodermic needle 114 with the first, sharp, engagement end covered before use by a cylindrical, plastic, removable sheath 116. The second end of the needle is covered by a connecting end 18 which attaches to the needle part way down the shaft. This connector is substantially a cylindrical tube, closed around the needle shaft at one end and open at a second end. The inner wall of the connector has a threaded insert. The needle extends from the closed end of the connector towards the open end of the connector. In this arrangement an injector pen 120 can screw into the open end of the connector and the second end of the needle is in fluid connection with the insulin within the pen.

FIG. 2 shows the inner configuration of the needle casing 100. The wide portion 102 provides a first housing portion, wide enough to receive the connecting end 118 of a needle assembly 112, onto which one end of a needle 114 is mounted. The narrow portion 104 of the needle casing has two portions. Firstly, a second housing portion 202, adjacent to the first housing portion, is too narrow to receive the connecting end 118 of the needle assembly 112, but can receive the needle 114 in the needle sheath 116. There is a second narrowing 204 separating this second housing portion 202 from a needle cavity 206. The needle cavity is too narrow to receive the needle sheath 116, but can receive the bare needle 114.

FIG. 2 additionally shows an internal projecting edge 208, extending radially inwards from the lip 110 at the open end 106 of the needle casing 100. This projecting edge 208 has beveled edges, sloping from the lip 110 into the casing. This projecting edge 208 causes the opening into the first housing portion 102 to be slightly narrower than the connecting end 118 of the needle assembly 112. The sloped edges 210 of the projecting edge 208 allow the connecting end 118 of the needle assembly 112 to pass over the projecting edge 208 when the needle assembly 112 is pushed into the needle casing 100, as the walls of the casing are pushed out to accommodate it. However once the connecting end 118 of the needle assembly 112 has passed fully over the projecting edge 208, it cannot pass back over the flat underside 212 of the projecting edge 208. It is retained within the needle casing 100.

Before the needle 114 is used and the needle sheath 116 is still covering it, the needle assembly 112 cannot pass far enough into the casing 100 for the connecting end 118 to pass fully over the projecting edge 208, (see FIG. 2a). However, after use (see FIG. 2b), when the needle 114 is exposed and can fit into the needle cavity 206, the needle assembly 112 can pass far enough into the casing 100 to pass fully over the projecting edge 208. Therefore in this configuration, before use the needle assembly 112 is insertable and removable from the casing 100, however after use once it is retained within the casing 100 below the projecting edge 208, it can no longer be removed. The casing 100 can then be disposed of, with the needle 114 inside, and there is no need for the user to come into direct contact with the used needle 114.

In order to allow for this safe use of the needle casing 112, when stored in a container, it must be held securely enough in the container to allow for the needle 114 to be screwed to an injector pen 120, removed and replaced. To assist with the screwing of the injector pen 120, the needle casing 112 must not rotate within the container 100. To assist with the removal and replacement of the needle 114 within the casing 100, the casing must not be accidentally removable from the container.

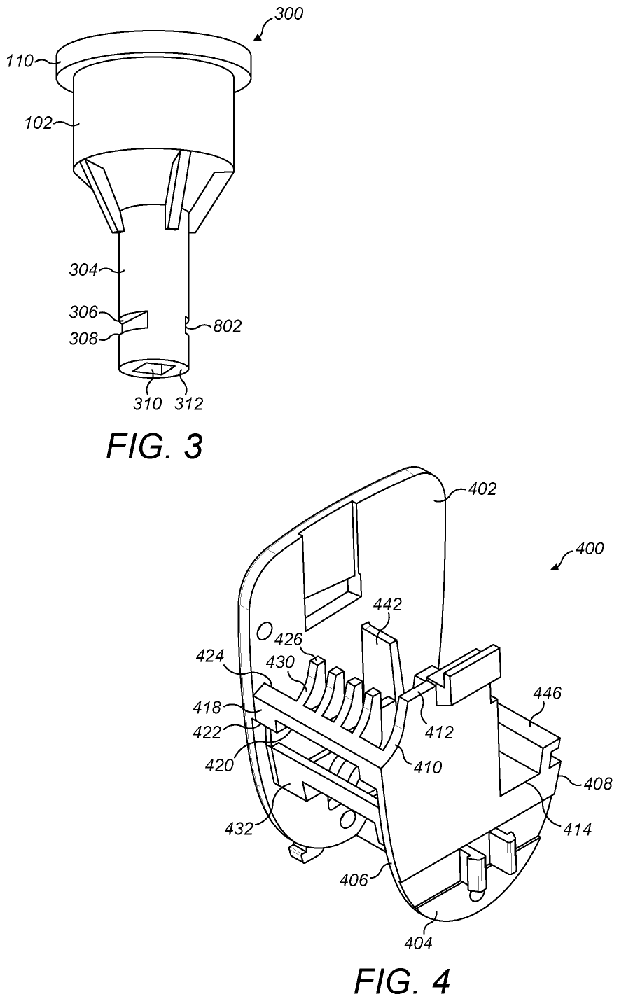

FIG. 3 shows the two features of the needle casing 300 that ensure that the needle casing 300 cannot rotate within or be removed from the container. The first feature is slots 302 in the side of the narrow portion 304 of the casing 300. Two slots 302 on opposite sides of the casing 300 can be used, or a single slot 302, cutting off a single portion of the circumference of the casing 300. Two, opposing slots 302 are used. They are located in the outer circumference of the casing 300, at a height equivalent to the needle cavity 206 inside the casing 300 where the housing material is thicker. The slots 302 create an upper 306 and lower surface 308 in the needle casing 300, as discussed further below.

The second feature of the needle casing 300 that prevents it rotating within the container is a square socket 310 in the closed end 312 of the casing 300. The closed end 312 of the needle casing 300 includes a square groove 310 that extends into the needle casing 300, but not as far as the closed end of the needle cavity 206.

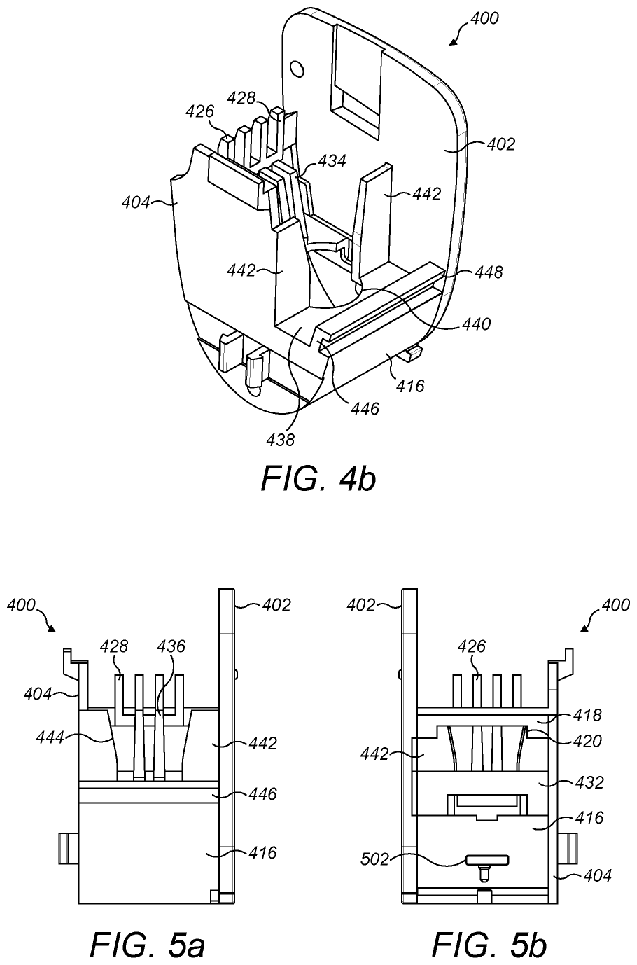

FIG. 4a shows the general assembly of the container 400 that receives the needle casing 100. The container 400 has two substantially planar first 402 and second 404 side walls that are positioned opposite each other in parallel. They both have curved bases 105. The first side wall 402 is substantially symmetrical about a central vertical axis running from the curved base 105 to the top edge, and extends the full height of the container 400. The second side wall 404 extends part of the way to the full height of the container 400 at the front edge 406 and has a lower rear edge 408 (see FIG. 4a). The top edge 410 of the second wall 404 angles upwards from the point at which it meets the front edge 406, then flattens out to form a flat section 412 before including a step down in height substantially halfway across the width of the wall, to create a second flat section 414 at the height of the rear edge 408 of the second wall 404, and extends horizontally to the point at which it meets the rear edge 408.

The two side walls 402, 404 are connected along a curved base by a base wall 416, better seen in FIG. 4b. This base wall 416 is a rectangular sheet of plastic curved along the base of the two side walls 402, 404 to connect them. The front side of the container additionally includes a front wall 418 connecting the two side walls 402, 404 near the top only. This front wall 418 has a rectangular notch 420 cut in the lower edge 422. Upwards from the inner edge 424 of the front wall extend four vertical protrusions 426. These four protrusions 426 provide flat faces 428 inside the container 400 and have curved front edges 430 such that they are narrow at the top and widest at the point where they meet the front wall 418.

Beneath the front wall 418, and set in from the edges of the two side walls 402, 404 is a bar 432 that extends between the side walls 402, 404 at the front of the container 400. This bar 432 has a flat top from which extend two vertical bars 434 extending between the front bar 432 and the front wall 418, and extending into the container 400. These vertical bars 434 form part of the internal structure used to aid in the secure storage of a needle casing 100. These can be seen more clearly in FIG. 5a. The flat inner faces 436 of the vertical bars 434 are angled such that they abut at least part of the substantially conical needle casing 100. Also forming part of the internal structure of the container 400, the sheet of material that forms the base wall 416, at its highest point up the rear side of the container 400, bends into the container at approximately 90° forming a ledge 438 that extends the full width of the container 400 between the two side walls 402, 404. This ledge 438 extends part way into the container, and the end provides a semi-circular cut-out 440. Either side of this cut-out 440, the ledge 438 then extends upwards to form two guides 442 up the side walls 402, 404 of the container 400. These guides 442 are flat sheets of the same material as the base wall 416 that each extends part of the way up the side walls 402, 404 of the container 400 from the ledge 428. They have angled inner edges 444, similar to the inner edges 436 of the vertical bars 434, also to abut at least part of the substantially conical needle casing 100 when inserted. The needle casing 100, when inserted, slots into the semi-circular cut-out 440 at the end of the ledge 438 of the base wall 416. Together, the ledge 438, the guides 442 and the vertical bars 434 act to guide the conical needle casing 100 into position in the container 400, and hold it in position.

There is a lower shelf 502 present in the container 400, protruding from the base wall 416, (see FIG. 5b). This lower shelf 502 is positioned below the lowest point of the needle casing 100 when it is inserted into the container 400, and extends over halfway across the inside of the container 400, such that the closed end 108 of the needle casing 100 can rest on it. At the rear of the ledge 438, where it meets the base wall 416, is a catch 446, protruding upwards from the ledge 438. This catch 446 extends upwards and is bent at approximately 90° at its highest point to create a tab 448, such that it can act as a simple catch, as explained in more detail below.

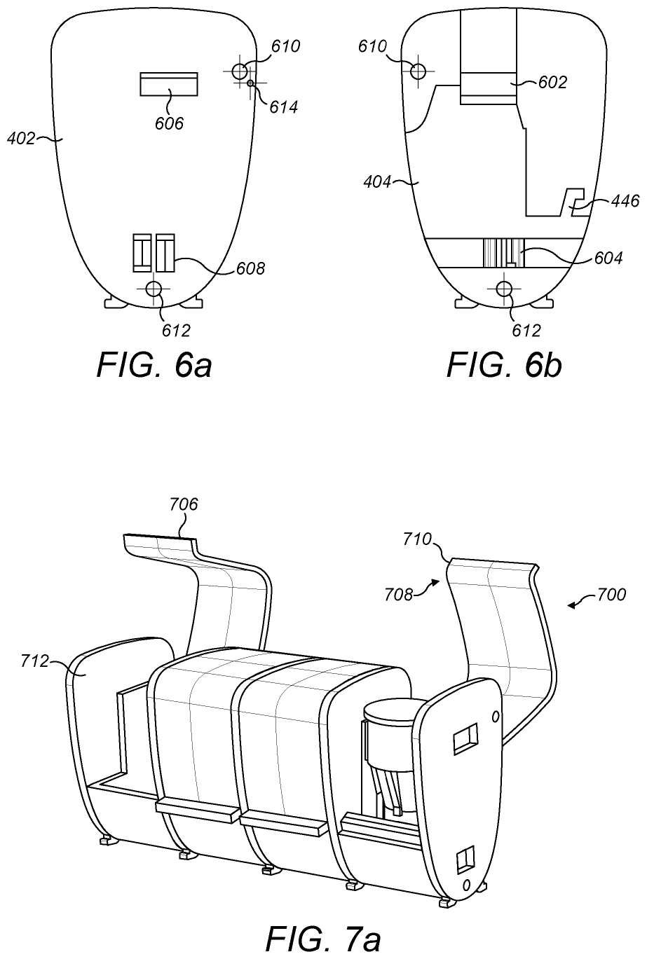

FIGS. 6a and 6b show the container from each end view. Male 602, 604 and female connectors 606, 608 can be seen on the two side walls 402, 404, which do not form part of the present disclosure. Two pivot holes can also be seen in the side walls 402, 404. At the top of the first side wall 402 is a pivot hole 610 for an access door (see FIG. 7a) of the container 400, described in more detail below. At the bottom of both side walls 402, 404 is a pivot hole 612 for a clamping element of the container 400, described in more detail below. Additionally, FIG. 6a shows a pip 614 used to hold the main access door open. This pip 614 is a circular projection extending into the compartment from the inner face of the first side wall 402. The edge of the access door closest to the first side wall 402 can only pass over this pip 614 if a force is applied by the user. The access door can therefore be held open if the door is pushed open to the point at which one edge of the door passes over the pip 614, and the access door is then retained on one side of the pip 614 in the open position.

FIGS. 7a and 7b show the access doors 700 of the needle container 400 that extend between the first 402 and second side walls 404 to form an enclosed compartment.

This access door 700 of the container 400 is made from a rigid but transparent material, such as polycarbonate. It is a rectangular sheet of material, curved to follow the contours of the side walls 402, 404 of the container. At one, first end 702 of the access door 700, the sheet material bends over on itself to create a circular gap (not shown) that extends the full width of the access door 700. This circular gap is configured to receive a pole (not shown) that acts as an axle, extending out of the circular gap 702 at each end. The ends of this axle are received in the pivot hole 610 of the first side wall 402 of each container 400. The axle pole can rotate within the pivot hole 610, allowing the access door 700 to pivot between an open position and a closed position.

At the second end 704 of the hinged door 700, the sheet of the door is bent at substantially 90° to create a handle 706. The inner face of the door becomes the bottom face of the handle 706. On this inner face of the door, positioned above the bend is a rectangular groove 708 that extends across the width of the door 700. The inner face 710 of the door below the groove 708 engages with the tab 448 of the catch 446 of each container. Due to the flexible nature of the polycarbonate, as the access door 700 is lowered into the closed position, the tab 448 pushes the inner face 710 outwards, such that it slides over the tab 448 as the door 700 is lowered further, until the point at which the tab 448 is retained within the groove 708. Once the tab 448 is positioned in the groove 708, the lower part of the inner face 710 cannot pass back over the tab 448. In this way the door is retained in the closed position.

The access door 700 is opened by applying pressure to the bottom face of the handle 706, bending the door 700 slightly, such that the flat inner face 710 of the door 700 can pass over the tab 448. In this arrangement the hinged door can be repeatedly opened and sealed.

At the front of the container 400, towards the base, between the two side walls 420, 404 is a hinged clamping element 800. This clamping element 800 (see FIG. 8), is substantially formed of a rectangular sheet of rigid plastic, made from a material such as Acetyl. At a first end of the clamping element 800, the sheet of material is folded back on itself to the point where it meets the inner face 802 of the clamping element 800, creating a circular gap 804 that extends across the full width of the clamping element 800. This circular gap 804 is configured to receive a pole (not shown) that acts as an axle, extending out of the circular gap 804 at each end. The axle is retained in the lower pivot holes 612 of the two side walls 402, 404, as described above. The axle pole can rotate within the circular holes 612, allowing the clamping element 800 to pivot.

At the second end of the clamping element 800, the sheet of material is bent at right angles such that it extends into the container to create a projecting arm 806, as discussed in more detail below. At the corner between the clamping element 800 and the projecting arm 806, a lever 808 extends upwards from the clamping element 800. A first portion 810 of this lever extends upwards to a height slightly above the bar 432 of the container 400 and is then bent at right angles, such that it extends into the container 400. It is then bent substantially upwards and back on itself and extends in the opposite direction, out from the container. The overlapping section of the sheet forms a compressible tab 812 that is wider than the gap between the bar 432 and the front wall 418 of the container 400. The end of the lever 808 extends downwards, past the first portion 810 of the lever, then outwards away from the container to create the base of a substantially Z-shaped strip 814. The base portion 814 is thicker than the rest of the sheet material for ease of use. If the base portion 814 of the lever 808 is depressed, the tab 812 is compressed to a width at which it can pass through the gap between the bar 432 and the front wall 418 of the container 400.

In a closed position the edges of the clamping element 800 engage with the bottom and front edges of the two side walls 402, 404, up to the bottom edge 422 of the front wall 418. The lever 808 of the clamping element 800 is depressed, compressing the tab 812, such that the tab 812 is narrow enough to slide into a gap underneath the front wall 418, in the rectangular notch 420, but above the front bar 432. Once the lever 808 is released, the tab 812 expands and is wedged between the front wall 418 and the front bar 432. To open the clamping element 800, the lever 808 is depressed, which compresses the tab 812, allowing it to slide out of the gap between the bar 432 and the front wall 432. In this arrangement the clamping element 800 can be repeatedly opened and sealed.

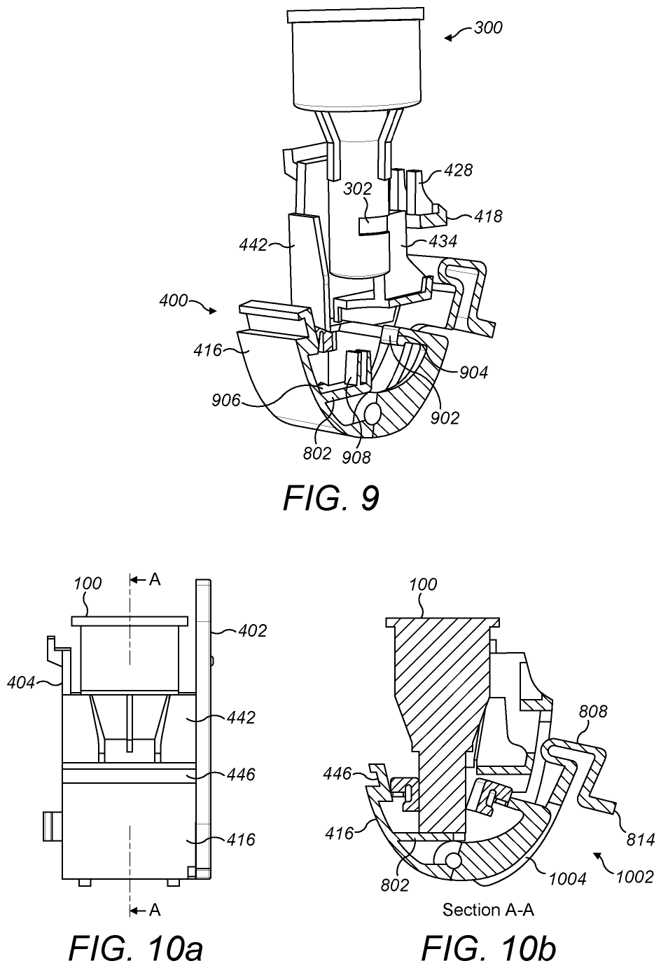

Extending from the inner surface of the clamping element 800 is a projecting arm 806, as seen in FIGS. 8 and 9. This arm 806, at its far end, away from the clamping element 800, has a concave cut-out 902. This leaves two teeth 904, one on either side of the arm. This arm 806 is configured to align with the slot 302 in the outer circumference of a needle casing 300 when it is located in the container 400. When the clamping element 800 is closed, the teeth 904 of the projecting arm 806 slide into the slots 302. The teeth 904 abut the upper 306 and lower 308 surfaces of the slots 302. In particular, as forces act on the needle casing 300 to pull it out of the container 400, the teeth 904 abut the lower surfaces 308 of the slots 302, preventing movement of the casing 300 out of the container 400. In this way, the needle casing 300 is retained in the container 400 when the needle assembly 112 inside the casing 300 is being removed for use. The casing 300 remains in this position until the needle 114 has been used and the needle assembly 112 returned to the needle casing 300. In order to dispose of the used needle 114, held securely in the needle casing 300, the hinged clamping element 800 is opened. The teeth 904 of the protruding arm 806 slide out of the slots 302, and when the container 400 is tipped, this needle casing 300 will fall out, ideally into a rubbish bin or similar. In this configuration there is no need for the user to come into contact with the used needle 114 to dispose of it.

On the upper side 906 of the lower shelf 502 that extends from the base wall 416 is a square plug 908. This extends upwards from the lower shelf 502 and is configured to engage with the square socket 310 in the closed end 312 of the needle casing 300. When a needle casing 300 is inserted into the container 400, the square plug 908 slides into the square socket 310 until the base 312 of the casing abuts the shelf 906 and it can move no further in. In use, when a user is twisting an injector pen 120 within the needle casing 300, either to attach to or release from the connecting end 118 of the needle assembly 112, the square socket 310 cannot rotate over the square plug 908. Therefore the needle casing 300 cannot rotate within the container 400.

In a second embodiment shown in FIGS. 10a and 10b an alternative clamping mechanism 1002 is used. This second clamping mechanism 1002 is suitable for the needle casings 100 of the first embodiment above and also for more commonly available commercial needle casings that do not have specially configured slots and sockets to aid retention in a container 400. This second clamping mechanism 1002 is incorporated into a container 400 similar to the container in the first embodiment above. It has side walls 402, 404 and a base wall 416, an access door through which to insert and access the needle casing 100, a front wall 418 and internal structure to support the needle casing 100 and a hinged clamping element 1004.

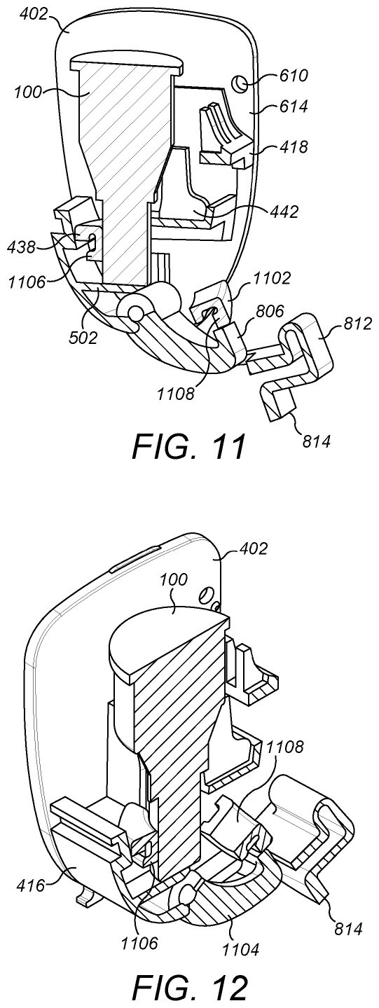

In this second embodiment two clamping jaws 1102 (see FIGS. 11 and 12), are used to retain the needle casing 100 in position. These clamping jaws 1102 are formed from a high friction material such as low surface energy rubber in order to grip the needle casing 100 most effectively. Each clamping jaw 1102 has a concave engaging surface 1104 configured to receive the curved outer wall of a needle casing 104. In this way the clamping jaws 1102 are in contact with the needle casing 100 over a large surface area to increase the grip the jaws 1102 have on the casing 100. A first clamping jaw 1106 is located on the ledge 438 that extends across the inner cavity of the container from the base wall 416. At the end of ledge 438 away from the base wall 416 of the container 400 the first clamping jaw 1106 is positioned, such that it can engage with the rear side of a needle casing 100. This first clamping jaw 1106 may either replace the semi-circular cut-out 440 used in the first embodiment or may be positioned over it. A second clamping jaw 1 108 is positioned on the projecting arm 806 of the clamping element 1004, at the far end of the arm, away from the clamping element 1004. In commercial needle casings 100 there is no socket 310 at the base 108 of the needle casing 100 and therefore there is no plug 908 present on the lower shelf 502 of the container 400 in this second embodiment.

The mechanism to retain the needle casing in place in the container 400 is the same as in the first embodiment of the present disclosure. The needle casing 100 is inserted into the container 400 until the base 108 of the casing 100 abuts the lower shelf 502 and cannot move any further. An area of the narrow portion 104 of the casing 100 is in contact with the first clamping jaw 1106. The clamping element 1004 is then pivoted to a point where it closes. At this point the second clamping jaw 1108 on the protruding arm 806 of the clamping element 1104 engages with the needle casing 100, opposite the first clamping jaw 1106. The needle casing 100 is retained in position between these two clamping jaws 1102.

In both of these embodiments the disposal mechanism for disposing of used needles 114 is the same. The needle assembly 112, once used, as detailed above, is retained within the needle casing 100. The casing 100 can therefore be tipped without exposing the needle 114. To dispose of the needle casing 100 from within the container 400 the clamping mechanism must be released. For both the first and second embodiments of the present disclosure this is done by depressing the base of the lever 814 on the hinged clamping element 1102 so that the tab 812 can slide out from within the container 400 and the clamping element 1102 is released. This removes the second clamping jaws 1108 on the protruding arms 806 from engagement with the needle casing 100. Therefore if the access door of the container 400 is opened and the container 400 is tipped upside down, the needle casing 100 will fall from the container 400 via gravity, without exposing the needle 114.

1. A system including a casing for shielding a hypodermic needle and a container for storing the casing,
the casing for shielding the hypodermic needle comprising:
a protective rigid housing comprised of a first open end, rigid side walls and a second closed end distal from the first open end,
wherein the first open end of the housing includes a first cylindrical inner cavity, configured to receive a first end of the hypodermic needle supported by a needle mounting assembly,
wherein the second closed end of the housing includes a cylindrical end portion, the cylindrical end portion having a second cylindrical inner cavity with a diameter smaller than the diameter of the first cylindrical inner cavity, and
wherein the second cylindrical inner cavity is configured to receive a second injection sharp end of the hypodermic needle, distal to the needle mounting assembly when the needle mounting assembly is received in the first cylindrical cavity, and
the container for storing the casing comprising:
a clamping mechanism, wherein an outer wall of the cylindrical end portion of the casing further comprises a fixing mechanism for engagement with the clamping mechanism within the container,
wherein the fixing mechanism comprises at least one slot in an outer wall of the cylindrical end portion, extending in a direction perpendicular to the longitudinal axis of the first and second cylindrical end portions, the slot being configured for engagement with the clamping mechanism; and
wherein the clamping mechanism includes a first end portion and a second end portion, wherein the first end portion is pivotably connected to a wall of the container and the second end of the clamping mechanism comprises a U-shaped clamp, such that the clamping mechanism is pivotable from a first position in which the casing within the container is not in contact with the U-shaped clamp, to a second position in which the U-shaped clamp is engaged with the at least one slot in the outer wall of the casing.

2. The system according to claim 1, wherein the fixing mechanism comprises two slots in an outer wall of the cylindrical end portion, both extending in a direction perpendicular to the longitudinal axis of the first and second cylindrical end portions.

3. The system according to claim 1, wherein the fixing mechanism includes two parallel slots on opposite sides of an outer wall of the cylindrical end portion of the casing, both extending in a direction perpendicular to the longitudinal axis of the first and second cylindrical end portions.

4. The system according to claim 1, wherein the outer wall of the rigid housing of the casing includes a frustroconical portion connecting the first open end of the housing and the second closed end.

5. The system according to claim 1, wherein the second cylindrical cavity of the casing comprises a narrowing to create a needle or sharps cavity with a diameter smaller than that of the second cylindrical cavity.

6. The system according to claim 5, wherein a needle sheath, covering a second injection sharp end of a hypodermic needle, distal to a needle mounting assembly supporting the needle has an outer diameter larger than the inner diameter of the needle or sharps cavity such that the needle sheath cannot pass into the needle or sharps cavity.

7. The system according to claim 1, wherein the first open end of the protective rigid housing of the casing includes a projecting edge with a bevelled first surface and a flat inner surface, extending radially inwards from at least one point on the circumference of the first open end.

8. The system according to claim 7, wherein the needle mounting assembly has a diameter larger than the narrowest diameter of the projecting edge, such that it cannot pass over the flat inner surface of the projecting edge.

9. The system according to claim 1, wherein the casing includes a removable lid to seal the first open end of the housing.

10. The system according to claim 1, wherein the second closed end of the housing includes a recession or socket.

11. The system according to claim 10, wherein the socket is non-circular.

12. The system according to claim 10, wherein the socket is substantially square.

13. A container for storing and/or shielding a casing for a hypodermic needle, said container including a rigid first wall and a rigid second wall opposing the first wall, the container further including a compartment for storing said casing, defined by the first and second walls configured to be end walls, a front wall and a rear wall extending between the first and second walls, an inner casing support structure, a pivotable access door and a pivotable clamping element;
the clamping element including a first end portion and a second end portion, wherein the first end portion is pivotably connected to a wall of the container and the second end of the clamping element comprises a substantially U-shaped clamp,
such that the clamping element is pivotable from a first position in which the casing within the container is not in contact with the U-shaped clamp, to a second position in which the U-shaped clamp is engaged with the casing.

14. A container according to claim 13, wherein the inner support structure includes a receiving surface for receiving the casing.

15. A receiving surface according to claim 14, wherein the receiving surface is substantially U-shaped.

16. A clamping element according to claim 13, wherein the clamping element includes an outer face that forms part of an outer wall of the container when the clamping element is in a first position, and an inner face that forms part of the inner wall of the container when the clamping element is in a first position.

17. A clamping element according to claim 16, wherein the clamping element includes a projecting arm, extending substantially perpendicularly from the inner face of the clamping element.

18. A clamping element according to claim 17, wherein the at least one U-shaped clamp is positioned on the projecting arm.

19. A container according to claim 13, wherein the container includes a first substantially U-shaped clamp on the clamping element and a second substantially U-shaped clamp on the inner support structure.

20. A container according to claim 19, wherein the second substantially U-shaped clamp is positioned for engagement with the casing at a point opposite the point at which the first U-shaped clamp on the clamping element engages the casing.

21. A container according to claim 13, wherein the U-shaped clamp is formed of rubber.

22. A system including a casing for shielding a hypodermic needle and a container for storing the casing,
the casing for shielding the hypodermic needle comprising:
a protective rigid housing comprised of a first open end, rigid side walls and a second closed end distal from the first open end,
wherein the first open end of the housing includes a first cylindrical inner cavity, configured to receive a first end of the hypodermic needle supported by a needle mounting assembly,
wherein the second closed end of the housing includes a cylindrical end portion, the cylindrical end portion having a second cylindrical inner cavity with a diameter smaller than the diameter of the first cylindrical inner cavity, and
wherein the second cylindrical inner cavity is configured to receive a second injection sharp end of the hypodermic needle, distal to the needle mounting assembly when the needle mounting assembly is received in the first cylindrical cavity, and
the container for storing the casing comprising the container according to claim 13.
